(19) United States
(12) Patent Application Publication
(10) Pub. No.: US 2007/0207197 A1
(43) Pub. Date: Sep. 6, 2007

(54) Composition and method for reducing symptoms of breast engorgement

(76) Inventors: J.A. Villarreal, Corpus Christi, TX (US); Noe Lira, Corpus Christi, TX (US); Thelma Lira, Corpus Christi, TX (US); Yolanda Villarreal, Corpus Christi, TX (US)

Correspondence Address: T. Ling Chwang, Suite 6000, 901 Main Street, Dallas, TX, 75202, US

(21) Appl. No.: 11/704,434

(22) Filed: Feb. 9, 2007

Related U.S. Application Data
(60) Provisional application No. 60/772,654, filed on Feb. 13, 2006.

(51) Int. Cl. A61K 9/127; A61K 36/31

(52) U.S. Cl. 424/450; 424/755

(57) ABSTRACT

A composition and method used to reduce the symptoms associated with breast engorgement, galactorrhea, and other associated disorders. More specifically, this invention is related to a cabbage extract combined with a base cream having liposomes that are applied to each breast of a patient suffering from a symptom of breast engorgement, galactorrhea, and other associated disorders.

This application claims priority to U.S. Provisional Patent Application, Ser. No. 60/772,654, entitled “Composition and method for reducing symptoms of breast engorgement” filed on Feb. 13, 2006, having J. A. Villarreal MD, Noe Lira MD, Thelma Lira MD, and Yolanda Villarreal RPN listed as the inventors, the entire content of which is hereby incorporated by reference.

### STATEMENT OF RIGHTS TO INVENTIONS MADE UNDER FEDERALLY SPONSORED RESEARCH

This invention not supported by any governmental grants.

### BACKGROUND

This invention is related to a composition and method that is used to reduce the symptoms associated with breast engorgement, galactorrhea, and other associated disorders. More specifically, this invention is related to a cabbage extract combined with a base cream having liposomes that are applied to each breast of a patient suffering from a symptom of breast engorgement, galactorrhea, and other associated disorders. Although not wanting to be bound by theory, the cabbage extract was incorporated into the liposome before being applied to the breast of the patient.

Common problems of breastfeeding occur in the postpartum period. Breastfeeding a healthy infant is often accompanied by many challenges. Some may be predictable, while others are unexpected. These problems may accompany the normal anxiety of first-time parents, who rarely appreciate the intensity of care infants require. Reassurance and guidance often will enable mothers to continue breastfeeding through the first year. However, common problems associated with breastfeeding in the postpartum period may lead to painful breasts, which, if untreated, is a common reason the some mothers stop breastfeeding in the first few weeks after delivery.

Engorgement. One specific problem associated with breastfeeding is called “ENGORGEMENT.” Engorgement refers to swelling of the breast and can occur early or late in the postpartum period. Early engorgement is secondary to edema, tissue swelling, and accumulated milk, while late engorgement is due solely to accumulated milk. Early engorgement accompanies the onset of copious milk production, also known as lactogenesis stage II. This typically occurs between 24 and 72 hours postpartum, with a normal range of one to seven days.

Early engorgement resolves spontaneously in the majority of cases, but may be exaggerated if the infant does not latch on well and nurse frequently and efficiently. Latching on refers to the formation of a tight seal of the infant's lips around the nipple and a sufficient portion of contiguous mammary tissue to allow efficient removal of milk during nursing. Poor latch-on interferes with the infant's ability to empty the breast. During engorgement, swelling of the breast tissue and reduced protractility of the nipple can make latch-on difficult.

Engorgement can be quite painful for some women, whose breasts become hard and warm to the touch. Frequent emptying of the breast can help to both prevent and treat engorgement. The initial step is to ensure that the infant can achieve a satisfactory latch-on. This may require softening the areola by hand expression of milk. Thereafter, frequent and thorough breastfeeding is mandatory.

Treatments for breast engorgement were evaluated by the Cochrane database in a systematic review of eight trials involving 424 women (Snowden, et al., Treatments for Breast Engorgement During Lactation. Cochrane Database Syst Rev 2001; CD000046).

The anti-inflammatory agent serrapeptase (Danzen) (OR 3.6, 95% confidence interval 1.3-10.3) and bromelain/trypsin complex (OR 8.02, 95% Cl 2.8-23.3) improved symptoms of engorgement, compared to placebo. Serrapeptase is available as an over-the-counter nutritional supplement. No data are available regarding its entry into milk or potential side effects in breastfeeding infants.

Massage appears to play a role in relieving discomfort. This was suggested by a randomized masked trial in 39 women in which application by massage of cabbage leaf extract (not otherwise effective) and placebo provided equivalent symptomatic relief (Roberts, et al., Effects of Cabbage Leaf Extract on Breast Engorgement. J Hum Lact 1998; 14:231).

The general recommendations for the relief of symptomatic breast engorgement are to complete emptying of the breasts at each feeding. Massage may help soften the breast and facilitate latching on.

Symptomatic relief may be obtained with cool compresses or ice packs. Ejection of milk between feedings may also be necessary. Mothers are advised to stand in a warm shower several times a day; allowing the spray to fall on the breasts often promotes milk release.

Mild analgesics such as acetaminophen or ibuprofen may provide effective pain management. These are considered safe in breastfeeding women by the American Academy of Pediatrics Committee on Drugs (Transfer of Drugs and Other Chemicals into Human Milk. Pediatrics 2001; 108:776). However, anti-inflammatory drugs available in the United States have not been tested in clinical trials for this indication.

Use of breast pumps for more than 10 minutes at a time should be avoided. Pumps are often inefficient for removing milk during early engorgement. Furthermore, the additional stimulation can exacerbate engorgement by promoting excess milk production. When pumps are used during engorgement, concurrent hand massage may help promote rapid milk removal.

Ice packs should be used with caution for engorgement in the early postpartum period. They provide temporary relief from discomfort, but can exacerbate swelling (Berens, P D. Prenatal, Intrapartum, and Postpartum Support of the Lactating Mother. Pediatr Clin North Am 2001; 48:365).

Late engorgement—Engorgement that occurs later in lactation is usually due to milk stasis rather than tissue swelling and may be generalized or limited to a single lobe of the breast. It can result from missed feedings or failure to empty the breast thoroughly. Treatment is similar to that for early engorgement, with cool compresses and massage, and warm showers to help express milk between feedings. However, heat packs may be used because tissue swelling usually is not present.

According to folklore, the application of cabbage leaves to the breast can reduce the discomfort of breast engorgement during weaning, but controlled studies indicated that cabbage leaves are not effective for this purpose. For example, cabbage leaves or cabbage leaf extracts, ultrasound treatments, and oxytocin were not beneficial, compared to routine care. In one particular study, the effectiveness of cabbage leaf extract was compared with that of a placebo in treating breast engorgement in lactating women. In a double-blind experiment with a pretest/posttest design, 21 participants received a cream containing cabbage leaf extract, while 18 received placebo cream. The placebo group received equal relief to the treated group, with the two groups showing no difference on all outcome measures. However, mothers perceived both creams to be effective in relieving discomfort. Feeding had a greater effect than the application of cream on relieving discomfort and decreasing tissue hardness. Thus, the use of cabbage leaves or cabbage leaf extract to treat engorgement actually teaches away from acceptable medical practice. It is therefore been recommended that lactation consultants encourage mothers to breastfeed if possible to relieve the discomfort of breast engorgement.

Galactorrhea. Galactorrhea refers to a discharge of milk or milk-like secretion from the breast beyond six months postpartum in a non-breastfeeding woman. The secretion may be intermittent or persistent, spontaneous or expressible, and unilateral or bilateral (usually bilateral).

A variety of stimuli can result in galactorrhea (ie, milk production unrelated to pregnancy or nursing). In most cases, these stimuli cause hyperprolactinemia. The nipple discharge, although usually white or clear, may also be yellow, green, brown, or gray. Microscopic examination of the discharge for the presence of fat globules or staining the discharge to detect fat helps to confirm the diagnosis if it is uncertain.

Although not wanting to be bound by theory, chronic breast stimulation from manipulation by the woman, her partner, or clothing (e.g. a poorly fitting brassiere) may cause galactorrhea.

Some steroid hormone preparations, most commonly oral contraceptive pills, can promote prolactin release and milk secretion. The mechanism by which estrogen stimulates prolactin secretion appears to involve binding of estrogen to the estrogen receptor, which then binds to an estrogen response element on the prolactin gene in the lactotroph cell of the pituitary.

Although not wanting to be bound by theory, lactation can be inhibited by prolactin-inhibiting factors, primarily dopamine released from the hypothalamus. Thus, drugs that inhibit dopamine action (eg, phenothiazines, methyldopa) or affect metabolism of other neurotransmitters in the brain can also result in milk production. In addition, any disease in or near the hypothalamus or pituitary that interferes with secretion of dopamine or its delivery to the hypothalamus can cause hyperprolactinemia. Stresses such as trauma, surgical procedures, and anesthesia may also inhibit dopamine release and induce galactorrhea.

Postthoracotomy syndrome is an unusual disorder in which the healing chest wound simulates the effect of a suckling infant. It can be associated with an elevated prolactin concentration, breast pain, and milk production. A similar effect can be seen with other forms of chest wall injury, including burns, cervical spine lesions, and herpes zoster. Other causes of hyperprolactinemia may include lactotroph adenomas, hypothyroidism, and chronic renal failure.

Cabbage: There are at least a hundred different cabbage types growing throughout the world. Generally, cabbage (Brassica Oleracea) is a hardy, cool-season crop that has been used for centuries for food supply as well as medicinal effects. The many varieties of cabbage can be wildly dissimilar, but most have a short, broad stem and leaves or flowers that form a compact head. Green cabbage is the most common type of cabbage used by American and European, it is pale green in color and the leaves are usually tightly compacted. Savoy Cabbage has green-yellow, crinkled leaves, and is less compact than the green cabbage. Napa Cabbage is Chinese cabbage with pale green crinkled leaves, elongated compact shape and white stems. Bok Choy is also Chinese cabbage with dark green leaves and white stems; leaves are held together loosely. Red Cabbage is dark purple red in color and its leaves are coarser than green cabbage.

Other types of cabbage include: early cabbage types grow quickly and mature quickly, so they must be harvested quickly. For example, this is a list of the standard sort of early cabbage grown: Jingan; Derby Day (Golden Acre); Charmant; Ruby Ball; Julius. Autumn cabbage types have thin, tender, succulent leaves are not very cold hardy, and so for very late maturity in maritime climates, the Europeans developed extra hardy types (January King and Wivoy) with thicker, tougher leaves. Extra-vigorous, hybrid savoys are the easiest-to-grow type of cabbage. Examples include: Red Rodan; Danish Ballhead; Gloria; Bently; Rougette; January King; Savonarch; Wivoy. Another type of cabbage is the over winter cabbage types, examples include: Springtime; and First Early Market.

Generally, the smooth green variety of cabbage having a tight head are useful for making engorgement creams (e.g. Brassica olerances capitata). In contrast, the red, savoy, or Chinese (e.g. Brassica rapa type) are not particularly useful for as ingredients for engorgement creams.

The invention described herein is a composition containing cabbage extract in combination with dermatological creams that are useful with topical delivery of drug compound (e.g. Base, cream with liposomes). Additionally, a method is described that can be used to reduce or eliminate the symptoms of engorgement by applying the cabbage extract cream to the breasts of women suffering the symptoms of engorgement. Although not wanting to be bound by theory, cabbage extract can come from a wide variety of different cabbages, as described above. Additionally, in order for any composition to have an effect, it is necessary for the effective composition to reach an intended target. In the case of engorgement, the target would be likely the lactation glands in the breast.

Topical Delivery of Compounds. The topical delivery of compositions or drugs through the skin provides many advantages; primarily, such a means of delivery is a comfortable, convenient and noninvasive way of administering drugs. The variable rates of absorption and metabolism encountered in oral treatment are avoided, and other inherent inconveniences, e.g., gastrointestinal irritation and the like, are eliminated as well. Transdermal drug delivery also makes possible a high degree of control over blood concentrations of any particular composition or drug.

Skin is a structurally complex, relatively thick membrane. Molecules moving from the environment into and through intact skin must first penetrate the stratum corneum and any material on its surface. They must then penetrate the viable epidermis, the papillary dermis, and the capillary walls into the blood stream or lymph channels. To be so absorbed, molecules must overcome a different resistance to penetration in each type of tissue. Transport across the skin membrane is thus a complex phenomenon. However, it is the cells of the stratum cornuem, which present the primary barrier to absorption of topical compositions or transdermally administered compositions or drugs. The stratum cornuem is a thin layer of dense, highly keratinized cells approximately 10-15 microns thick over most of the body. It is believed to be the high degree of keratinization within these cells as well as their dense packing that creates in most cases a substantially impermeable barrier to composition or drug penetration. With many compositions or drugs, the rate of permeation through the skin is extremely low without the use of some means to enhance the permeability of the skin.

Numerous chemical agents have been studied as a means of increasing the rate at which a composition or drug penetrates through the skin. As will be appreciated by those in the field, chemical enhancers are compounds that are administered along with the composition or drug (or in some cases the skin may be pretreated with a chemical enhancer) in order to increase the permeability of the stratum cornuem, and thereby provide for enhanced penetration of the composition or drug through the skin. Ideally, such chemical penetration enhancers or “permeation enhancers,” as the compounds are referred to herein, are compounds that are innocuous and serve merely to facilitate diffusion of the composition or drug through the stratum corneum. The permeability of many therapeutic agents with diverse physicochemical characteristics may be enhanced using these chemical enhancement means. However, there are skin irritation and sensitization problems associated with high levels of certain enhancers.

Base compositions. One method of delivering an active agent through the skin is to utilize formulations having a basic pH. While the method of delivery of an active agent may vary, the method will typically involve application of a formulation or drug delivery system containing a pharmaceutically acceptable inorganic or organic base to a predetermined area of the skin or other tissue for a period of time sufficient to provide the desired local or systemic effect. The method may involve direct application of the composition as an ointment, gel, cream, or the like, or may involve use of a drug delivery device. In either case, water is preferably present in order for the hydroxide ions to be provided by the base, and thus enhance the flux of the active agent through the patient's body surface. Thus, such a formulation or drug reservoir may be aqueous, i.e., contain water, or may be non-aqueous and used in combination with an occlusive backing so that moisture evaporating from the body surface is maintained within the formulation or transdermal system during drug administration. In some cases, however, e.g., with an occlusive gel, a nonaqueous formulation may be used with or without an occlusive backing layer.

Suitable formulations include ointments, creams, gels, lotions, solutions, pastes, and the like with or without liposomes (as described below). Ointments, as is well known in the art of pharmaceutical formulation, are semisolid preparations that are typically based on petrolatum or other petroleum derivatives. The specific ointment foundation to be used, as will be appreciated by those skilled in the art, is one that will provide for optimum drug delivery, and, preferably, will provide for other desired characteristics as well, e.g., emolliency or the like. As with other carriers or vehicles, an ointment foundation should be inert, stable, nonirritating and nonsensitizing. As explained in Remington: The Science and Practice of Pharmacy, 20th edition (Lippincott Williams & Wilkins, 2000), ointment foundations may be grouped in four classes: oleaginous, emulsifiable, emulsion, and water-soluble. Oleaginous ointment foundations include, for example, vegetable oils, fats obtained from animals, and semisolid hydrocarbons obtained from petroleum. Emulsifiable ointment foundations, also known as absorbent ointment foundations, contain little or no water and include, for example, hydroxystearin sulfate, anhydrous lanolin and hydrophilic petrolatum. Emulsion ointment foundations are either water-in-oil (W/O) emulsions or oil-in-water (O/W) emulsions, and include, for example, cetyl alcohol, glyceryl monostearate, lanolin and stearic acid. Water-soluble ointment foundations can be prepared from polyethylene glycols of varying molecular weight.

Liposomes: The delivery of drugs or compounds to the skin and systemically via the skin is hampered by the natural barrier of the stratum corneum. Creams and lotions are classical vehicles for delivering drugs and cosmetics to the skin. Generally, these preparations are semi-solid, bi-phasic preparations where oil spheres are dispersed in water. The droplet size of these spheres have not been a concern in conventional pharmaceutically marketed semi-solid creams and lotions. Most commercially marketed medical creams include oil spheres having a size of 5 to 50 microns. Additionally, liposomes have been used as useful delivery vehicles for compositions or drugs through the skin.

Liposomes are lipid vesicles composed of membrane-like lipid layers surrounding aqueous compartments. Liposomes are widely used to encapsulate biologically active materials for a variety of purposes, but particularly they are used as drug carriers. Depending on the number of lipid layers, size, surface charge, lipid composition and methods of preparation various types of liposomes have been utilized.

Multilamellar lipid vesicles (MLV) were first described by Bangham, et al., (J. Mol. Biol. 13: 238:252, 1965). A wide variety of phospholipids form MLV on hydration. MLV are composed of a number of bimolecular lamellae interspersed with an aqueous medium. The lipids or lipophilic substances are dissolved in an organic solvent. The solvent is removed under vacuum by rotary evaporation. The lipid residue forms a film on the wall of the container. An aqueous solution generally containing electrolytes and/or hydrophilic biologically active materials are added to the film. Agitation produces larger multilamellar vesicles. Small multilamellar vesicles can be prepared by sonication or sequential filtration through filters with decreasing pore size. Small unilamellar vesicles can be prepared by more extensive sonication. An improved method of encapsulating biologically active materials in multilamellar lipid vesicles is described in U.S. Pat. No. 4,485,054.

Multilamellar vesicles can be reduced both in size and in number of lamellae by extrusion through a small orifice under pressure, e.g., in a French press. The French press (Y. Barenholz; S. Amselem and D. Lichtenberg, FEBS Lett. 99: 210-214, 1979), extrusion is done at pressures of 20,000 lbs/in at low temperature. This is a simple, reproducible, non-destructive technique with relatively high encapsulation efficiency, however it requires multilamellar liposomes as a starting point that could be altered to oligo- or unilamellar vesicles. Large unilamellar lipid vesicles (LUV) can be prepared by the reverse phase evaporation technique (U.S. Pat. No. 4,234,871, Papahadjopoulos). This technique consists of forming a water-in-oil emulsion of (a) the lipids in an organic solvent and (b) the substances to be encapsulated in an aqueous buffer solution. Removal of the organic solvent under reduced pressure produces a mixture which can then be converted to the lipid vesicles by agitation or by dispersion in an aqueous media.

U.S. Pat. No. 4,016,100, Suzuki, et al., describes still another method of entrapping certain biologically active materials in unilamellar lipid vesicles by freezing an aqueous phospholipid dispersion of the biologically active materials and lipids. All the above liposomes, made prior to 1983, can be classified either as multilamellar or unilamallar lipid vesicles. A newer type of liposomes is referred to as multivesicular liposomes (S. Kim, M. S. Turker, E. Y. Chi, S. Sela and G. M. Martin, Biochim. Biophys. Acta 728; 339-348, 1983). The multivesicular liposomes are spherical in shape and contain internal granular structures. A lipid bilayer forms the outermost membrane and the internal space is divided up into small compartments by bilayer septrum. This type of liposomes required the following composition: an amphiphatic lipid with net neutral charge, one with negative charge, cholesterol and a triacylglycerol. The aqueous phase containing the material to be encapsulated is added to the lipid phase that is dissolved in chloroform and diethyl ether, and a lipid-in-water emulsion is prepared as the first step in preparing multivesicular liposomes. Then a sucrose solution is shaken with the water-in-lipid emulsion; when the organic solvents are evaporated liposomes with multiple compartments are formed.

For a comprehensive review of types of liposome and methods for preparing them, refer to a recent publication “Liposome Technology” Ed. by G. Gregoriadis. CRC Press Inc., Boca Raton, Fla., Vol. I, II, & III 1984.

Liposomes are microscopic, fluid-filled pouch whose walls are made of layers of phospholipids identical to the phospholipids that make up cell membranes. Liposomes have been used to deliver certain vaccines, enzymes, or drugs (e.g., insulin and some cancer drugs) to the body. When used in the delivery of certain cancer drugs, liposomes help to shield healthy cells from the drugs' toxicity and prevent their concentration in vulnerable tissues (e.g., the kidneys, and liver), lessening or eliminating the common side effects of nausea, fatigue, and hair loss. Liposomes are especially effective in treating diseases that affect the phagocytes of the immune system because they tend to accumulate in the phagocytes, which recognize them as foreign invaders. They have also been used experimentally to carry normal genes into a cell in order to replace defective, disease-causing genes. Liposomes are sometimes used in cosmetics because of their moisturizing qualities. Liposomes were first produced in England by Alec D. Bangham, who was studying phospholipids and blood clotting. It was found that phospholipids combined with water immediately formed a sphere because one end of each molecule is water soluble, while the opposite end is water insoluble. Water-soluble medications added to the water were trapped inside the aggregation of the hydrophobic ends; fat-soluble medications were incorporated into the phospholipid layer.

Generally, many compounds can be easily incorporated in liposomes. Although not wanting to be bound by theory, one must remember the that the penetration rate vs. particle size has a logarithmic relationship in that a particle that is half the size is likely to have far higher than double the rate of penetration. Generally, an ideal drug candidates for transdermal application include:
1. non-ionic (polarity);
2. low molecular weight or less than 500 Daltons;
3. have adequate solubility in oil and water (log P in the range of 1 to 3);
4. a low melting point (less than 200° C.);
5. are potent or dose is less than 50 mg per day;

Some of the other factors to consider include:
1. application area or mg per sq. cm.;
2. solubility of actives;
3. interval of application (how often apply the formula);
4. thickness of skin (i.e. choose thin or thick area);
5. receptors (availability of capillaries)—like the keys and locks;
6. disease state of a patient; and
7. temperature.

One active liposome useful for this invention include a transparent microdispersion comprising lysophospholipids and perilla oil. This system provides a microemulsion delivery with ultra-small particles below 100 nm. It is derived from natural ingredients. One liposome useful for this invention comprises a highly penetrative lysophospholipids molecularly dispersed in hydrophilic medium. With Perilla seed oil, the mixture forms a system of phospholipids vesicles and micro oil-globules characterized by very high stability and ultra-small particles below 100 nm. The finely dispersed system is almost transparent. Although not wanting to be bound by theory, it is believed that these characteristics give rise to the formula's high affinity for skin and cell membranes.

Lysophospholipids are derived from natural lecithin by the same enzymatic process in humans for digestion of triglycerides. One of the two fatty acids of phospholipids is split off giving more hydrophilic properties to the molecule. Although not wanting to be bound by theory, this naturally engineered molecule exhibits useful properties such as emulsifying or dispersing properties and anti-irritant, but most of all improving absorption.

Perilla is Labiateae family member and is cultivated in Asia. The oil is directly extracted from the seed by cold-pressing. It is very rich in polyunsaturated essential fatty acids and particularly in alpha-linolenic acid. Although not wanting to be bound by theory, this vitamin-like molecule is an efficient cellular shield not only to reduce wrinkles and loss of elasticity but also to reinforce cutaneous tissues.

In a paper published by Kirjavainen M, et al., titled “Interaction of Liposomes With Human Skin In Vitro—The Influence of Lipid Composition and Structure,” Biochim Biophys Acta. 1996 Dec. 13; 1304(3):179-89, liposomes were suggested as a vehicle for dermal and transdermal drug delivery. However, the knowledge about the interaction between lipid vesicles and human skin is poor, so liposome penetration into the human skin was visualized by confocal laser scanning microscopy (CLSM) in vitro. Liposomes were prepared from phospholipids in different compositions and labeled with a fluorescent lipid bilayer marker, N—Rh-PE (L-alpha-phosphatidylethanolamine-N-lissamine rhodamine B sulfonyl). Fluorescently labeled liposomes were not able to penetrate into the granular layers of epidermis. The fluorescence from liposome compositions containing DOPE (dioleylphosphatidyl ethanolamine) was able to penetrate deeper into the stratum corneum than that from liposomes without DOPE. Pretreatment of skin with unlabeled liposomes containing DOPE or lyso-phosphatidyl choline (lyso-PC) enhanced the subsequent penetration of the fluorescent markers, N—Rh-PE and sulforhodamine B into the skin, suggesting possible enhancer activity, while most liposomes did not show such enhancement.

Resonance energy transfer (RET) and calcein release assay between stratum corneum lipid liposomes (SCLLs) and the phospholipid vesicles suggesting that the liposomes containing DOPE may fuse or mix with skin lipids in vitro and loosen the SCLL bilayers, respectively. Although not wanting to be bound by theory, the factors not affecting stratum corneum penetration were: negative charge, cholesterol inclusion and acyl chain length of the phospholipids. The Kirjavainen paper concluded that fusogenicity of the liposome composition appears to be a prerequisite for the skin penetration.

Although not wanting to be bound by theory, adding skin penetrants to a formula may enhance topical and transdermal delivery of very impermeable compounds. For example, one problem in topical and transdermal delivery is poor drug bioavailability. It is generally believed that only a few percent of a topically applied corticosteroid reaches its target site. In terms of delivery efficiency, which includes safety issues, it is preferable to deliver all drugs to the action site. This can be achieved by optimizing the formulation.

Although not wanting to be bound by theory, the most appropriate formulation components can be selected based on their physicochemical properties and an understanding of how penetrants cross the stratum corneum (SC), a significant barrier to skin penetration. Although not wanting to be bound by theory, to make the SC more permeable to an extended range of molecules, it may be necessary to employ penetration technologies, such as skin penetrants, iontophoresis, and sonophoresis to reversibly and safely modify percutaneous absorption.

Although not wanting to be bound by theory, many drugs are diffused through the intercellular channels and the route is tortuous. Considering the actual thickness of the SC, 15 mm, the path length is estimated between 350 and 1,000 mm. This is one reason the skin is extremely impermeable. Another reason is the nature of the lipids in the cell membrane. The lipids are composed of complex ceramides (50%), cholesterol (25%), free fatty acids (15%) and cholesteryl sulphate (5%); these will form well-structured bilayers. Therefore, a diffusing drug molecule has to transport across series of bilayers that form very rigid structures. The physiochemical features that control transferring ability include solubility, molecular size, shape and charge distribution of drug, and partition coefficient.

Fick's First Law of Diffusion describes the steady state of flux (J) across a membrane, showing the reasons the physiochemical characteristics need to be considered: J=D KCs/Ah where D is the diffusion coefficient, K is partition coefficient between skin and vehicle, Cs is drug concentration in the vehicle (mg/cm3), A is skin surface (cm2) and h is skin thickness (cm). The diffusion coefficient depends on molecular size and shape and will be related to the rigidity of the bilayer structures through which the drug has to pass. The partition coefficient depends on the affinity of the applied drug into skin and vehicle. Therefore, factors to consider in percutaneous absorption are diffusion coefficient, partition coefficient and solubility of the applied drug. It is probable that skin penetrants modify all three parameters.

U.S. Patent Application Publication US 2004/0258778A1 (Ser. No. 10/814,607), titled “Lactation Cessation and Breast Engorgement compositions and Methods of Use,” with Farmer et al., listed as inventors, was published on Dec. 23, 2004, (“the '607 Application”). The '607 Application describes a cabbage extract composition that is effective for acceleration lactation cessation in both breast-feeding and non-breastfeeding mothers. The cabbage extract composition was also found to prevent or alleviate the symptoms of bread engorgement in lactating mothers. The composition and methods described in the '607 Application do not disclose liposomes.

The invention described herein utilizes a composition of cabbage extract, base cream and a liposomes cream in a method that is useful to reduce the symptoms associated with breast engorgement, galactorrhea, and other associated disorders.

### SUMMARY

Breast engorgement refers to abnormal swelling of one or both breasts that can occur during early or late postpartum period. Early engorgement is secondary to edema, tissue swelling, and accumulated milk, while late engorgement is due solely to accumulated milk. Early engorgement accompanies the onset of copious milk production, also known as lactogenesis stage II, which typically occurs between 24 and 72 hours postpartum, with a normal range of one to seven days.

One aspect of the current invention is a composition used for resolving a symptom of breast engorgement in a patient suffering symptoms of breast engorgement. The composition is made using (a) extracted cabbage juice; and (b) base cream containing liposomes. The ratio of extracted cabbage juice in milliliters (“ml”) to base cream containing liposomes in grams (“gm”) is about 1 ml:30 gm. Other preferred ratios include: a ratio about 5 ml:30 gm; a ratio of about 10 ml:30 gm; a ratio of about 15 ml:30 gm; a ratio of about 20 ml:30 gm and a ratio of about 25 ml:30 gm.

A second aspect of the current invention is method of resolving a symptom of breast engorgement, galactorrhea, and other associated disorders in a patient suffering symptoms of breast engorgement, galactorrhea, and other associated disorders. The method involves: applying a composition containing cabbage juice and base cream having liposomes to a defatted portion of each breast of the patient, and repeating applications at least once a day until the symptom of breast engorgement, galactorrhea, and other associated disorders has been resolved. In preferred embodiments, the composition is applied at least two-three times a day until symptoms are resolved. The symptoms that can be resolved include: nipple leakage, breast pain, lactation cessation, galactorrhea and others. In a preferred embodiment, the application of the composition containing cabbage juice and base cream having liposomes repeating for about 7 to about 10 days.

A third aspect of the current invention involves, a method of resolving a symptom of inflammation or pain in a joint of a patient suffering symptoms of joint pain. The method involves: applying a composition containing cabbage juice and base cream having liposomes to the affected joint of the patient, and repeating applications at least once a day until the symptom of joint pain has been resolved. In preferred embodiments, the composition is applied at least two-three times a day until symptoms are resolved. The composition can be applied to knee joints, finger joints, hip joints, ankle joints, wrist joints and other joints. In a preferred embodiment, the application of the composition containing cabbage juice and base cream having liposomes repeating for about 1 to about 21 days.

### DETAILED DESCRIPTION OF PREFERRED EMBODIMENTS

Before describing the present invention in detail, it is to be understood that this invention is not limited to particular compositions or composition delivery systems, which may vary. It is also to be understood that the terminology used herein is for the purpose of describing particular embodiments only, and is not intended to be limiting. In addition, before describing detailed embodiments of the invention, it will be useful to set forth definitions that are used in describing the invention. The definitions set forth apply only to the terms as they are used in this patent and may not be applicable to the same terms as used elsewhere, for example in scientific literature or other patents or applications including other applications by these inventors or assigned to common owners. Additionally, when examples are given, they are intended to be exemplary only and not to be restrictive.

It must be noted that, as used in this specification and the appended claims, the singular forms “a,” “an” and “the” include plural referents unless the context clearly dictates otherwise. Thus, for example, reference to “a pharmacologically active agent” includes a mixture of two or more such compounds, reference to “a base” includes mixtures of two or more bases, and the like.

In describing and claiming the present invention, the following terminology will be used in accordance with the definitions set out below.

“Active agent,” “pharmacologically active agent,” “composition,” and “drug” are used interchangeably herein to refer to compositions and drugs that are useful for the treatment engorgement and joint pain relief. The terms also encompass pharmaceutically acceptable, pharmacologically active derivatives and analogs of such drugs, including, but not limited to, salts, esters, amides, prodrugs, active metabolites, inclusion complexes, analogs, and the like. Therefore, when the terms “active agent,” “pharmacologically active agent”, or “drug” are used, it is to be understood that applicants intend to include the active engorgement composition per se as well as pharmaceutically acceptable, pharmacologically active salts, esters, amides, prodrugs, active metabolites, inclusion complexes, analogs, etc., which are collectively referred to herein as “pharmaceutically acceptable derivatives.”

The term “aqueous” refers to a composition, formulation or drug delivery system that contains water or that becomes water-containing following application to the skin or mucosal tissue.

The term “base” is used in its traditional sense, i.e., a substance that dissolves in water to produce hydroxide ions. The water is typically an aqueous fluid, and may be natural moisture at the skin surface, or the patch or composition that is used may contain added water, and/or be used in connection with an occlusive backing. Similarly, any liquid or semisolid formulation that is used is preferably aqueous or used in conjunction with an overlayer of an occlusive material. Any base cream may be used provided that the compound provides free hydroxide ions in the presence of an aqueous fluid. Bases can provide free hydroxide ions either directly or indirectly and thus can also be referred to as “hydroxide-releasing agents”. Hydroxide-releasing agents that provide free hydroxide ions directly, typically contain hydroxide groups and release the hydroxide ions directly into solution, for example, alkali metal hydroxides. Hydroxide-releasing agents that provide free hydroxide ions indirectly, are typically those compounds that are acted upon chemically in an aqueous environment and the reaction produces hydroxide ions, for example metal carbonates or amines.

“Body surface” is used to refer to skin.

“Carriers” or “vehicles” as used herein refer to carrier materials suitable for transdermal or topical composition or drug administration. Carriers and vehicles useful herein include any such materials known in the art, which are nontoxic and do not interact with other components of the composition in a deleterious manner.

“Effective amount” or “a therapeutically effective amount” of a drug for the treatment of symptoms of engorgement is intended to mean a nontoxic but sufficient amount of the drug to provide the desired therapeutic effect. The amount that is “effective” will vary from subject to subject, depending on the age and general condition of the individual, the particular concentration and composition being administered, and the like. Thus, it is not always possible to specify an exact effective amount. However, an appropriate effective amount in any individual case may be determined by one of ordinary skill in the art using routine experimentation. Furthermore, the exact effective amount of the engorgement or joint pain composition incorporated into a composition or dosage form of the invention is not critical, so long as the concentration is within a range sufficient to permit ready application of the formulation so as to deliver an amount of the drug that is within a therapeutically effective range.

“Effective amount” or “an effective permeation enhancing amount” of a permeation enhancer refers to a nontoxic, non-damaging but sufficient amount of the enhancer composition to provide the desired increase in skin permeability and, correspondingly, the desired depth of penetration, rate of administration, and amount of engorgement or joint pain composition delivered.

“Penetration enhancement” or “permeation enhancement” as used herein relates to an increase in the permeability of the skin to the engorgement relief composition or drug, i.e., so that the rate at which the composition or drug permeates therethrough (i.e., the “flux” of drug through the body surface) is increased relative to the rate that would be obtained in the absence of the permeation enhancer.

“Predetermined area” of skin, refers to the area of skin through which a composition formulation is delivered, and is a defined area of intact unbroken living skin. That area will usually be in the range of about 5-800 cm², more usually in the range of about 50-400 cm², preferably in the range of about 100-200 cm². However, it will be appreciated by those skilled in the art of drug delivery that the area of skin through which drug is administered may vary significantly, depending on the individual, patch configuration, dose, and the like.

“Topical administration” is used in its conventional sense to mean delivery of a composition or drug for the treatment of engorgement to the skin, as in, for example, the treatment of various skin disorders. Topical administration, in contrast to transdermal administration, provides a local rather than a systemic effect. However, unless otherwise stated or implied, the terms “topical drug administration” and “transdermal drug administration” are used interchangeably.

“Transdermal” drug delivery is meant administration of a composition or drug for the treatment of engorgement to the skin surface of an individual so that the composition or drug passes through the skin tissue and into the individual's blood stream, thereby providing a systemic effect.

“Treating” and “treatment” as used herein refer to reduction in severity and/or frequency of symptoms, elimination of symptoms and/or underlying cause, prevention of the occurrence of symptoms and/or their underlying cause, and improvement.

The present invention pertains to compositions and methods of treating breast engorgement, galactorrhea, and other associated disorders using an effective amount of cabbage extract combined with a base cream having liposomes. More specifically, the inner leaves of a smooth green variety of cabbage were processed into extract, filtered and combined with a base cream having liposomes. The resultant mixture can then be applied to the breast of a patient having symptoms of engorgement, and leads to cessation of lactation in about 3-5 days.

### EXAMPLES

The following examples are provided to further illustrate this invention and the manner in which it may be carried out. It will be understood, however, that the specific details given in the examples have been chosen for purposes of illustration only and not be construed as limiting the invention.

### Example 1

The process, technique, and apparatus for synthesizing the NOLAC cream formula having and effective amount of about 5% cabbage extract in base cream with liposomes. The corresponding patient results are described below.

1.1 Materials: Cabbage juice extract was prepared using about one-half of a head of cabbage, more specifically the inner leaves of a smooth green cabbage head were used, and the cabbage head was not bruised. Generally, the white inner leaves were processed because the green outer leaves required extra filtration and light exposure to bleach the extract. The cabbage stem was not used. The cabbage was purchased at a local produce vender (San Antonio, Tex.). The inner leaves from one half of head of cabbage were processed using a juice processor (Juiceman Juicer®). Alternatively, juice processors that are manufactured by different companies such as a Green Star GS1000 Juicer, L'Equip Visor 509 Juicer, Miracle Business MJ575 Wheatgrass Juicer, and other suitable juice processors could be utilized or adapted for large scale manufacture. The cabbage juice extract was filtered twice using a Kerr-white filter paper 6″ size from (Apothecary Products) with a medium flow rate. Other filters known in the art could also be utilized or adapted for large scale manufacture. The filtered composition stood for about 48 hours until clear. In one embodiment, the filtered composition was colorless or naturally bleached liquid. Alternatively, the extract could be clarified by centrifugation and the supernatant used as the clear cabbage juice. One of ordinary skill in the art is familiar with filtering, centrifugation, and other clarifying techniques used in the art. Clarification techniques are very widespread and do not need to be discussed further.

About 5 ml of the filtered and cleared cabbage extract was mixed in about 15 grams of base cream with liposome. The liposomal No. B1204P base cream was purchased from Spectrum Pharmacy Products (Tucson, Ariz.). This base cream contains liposomes that are the smallest artificial vesicles of spherical shape that can be produced from natural nontoxic phospholipids and cholesterol, although other liposomes could also be utilized. Although not wanting to be bound by theory, the vesicles can be used as drug carriers that are extremely versatile and due to the variability of their composition can be used for a large number of applications. The clarified cabbage extract was incorporated into the liposomal base cream using 2 spatulas on a glass tile. Larger quantities incorporating a greater volume of liquid to reach the high percentages of extract in the cream utilized a stainless steel mixer bowl to mix the liposome cream base on a slow speed . The cabbage extract was added to the base cream slowly, ( e.g. about 5 ml at a time, and the cabbage extract was incorporated into the base cream having liposomes

An effective amount of cabbage extract and base cream with liposomes was used to resolve the symptoms of engorgement in patients suffering from the same. The resultant cream was then applied to the defatted clean surface of each breast for about 3 to 5 days. Although not wanting to be bound by theory, the composition of cabbage extract and liposomal base cream maximized the absorption of the natural ingredients contained in the cabbage extract thru the skin of the breast into the glands of lactation resulting in a decrease in lactation eventually ceasing in about 3 to 5 days. Additionally, suppression of lactation usually resulted in 3 to 5 days depending on the strength of the formula and/or the cause of the lactation.

As shown in Table 1, three different concentrations of cabbage extract cream were tested on patients suffering from symptoms of engorgement.

The composition was applied to an area of the breast around the nipple having an area of about 121 cm².

TABLE 1
Patient Results NOLAC Cream

| | | | |
|---|---|---|---|
| Patient # | Age | Symptoms/Diagnosis | Results/Resolution + or − |

Strength About 25%

| | | | | |
|---|---|---|---|---|
| #1 | 38 | Breast engorgement | + resolution | OB |
| #2 | 27 | Breast engorgement | + resolution | OB |
| #3 | 32 | Breast engorgement | + resolution | OB |
| #4 | 22 | Nipple Leakage | + resolution | GYN |
| #5 | 25 | Nipple Leakage | + resolution | OB |
| #6 | 27 | Breast engorgement | + resolution | OB |
| | | | substantial decrease | |
| #7 | 23 | Breast pain and tightness | + resolution after 1 day | OB |
| #8 | 37 | Lactation cessation | + resolution, no lactation after 1 day | OB |
| #9 | 30 | Galactorrhea | + resolution, major | GYN |
| #10 | 22 | Breast pain | + resolution, pain gone | OB |
| #11 | 26 | Lactation cessation | + resolution, after 3 days completely | OB |
| | | | dry | |
| #12 | 32 | Breast pain | + resolution pain gone after 1 day | OB |
| #13 | 36 | Breast pain and lactation | + resolution in 3 days in both areas | OB |
| | | cessation | | |
| #14 | 30 | Lactation cessation | + resolution | OB |

Strength About 5%

| | | | | |
|---|---|---|---|---|
| New | 5% | | | |
| Strength | | | | |
| #15 | 25 | Lactation cessation | + resolution in 21 days Weak response | OB |
| #16 | 29 | Lactation cessation | No response in 14 days, switch to 30%, | OB |
| | | | + resolution in 2 days | |
| #17 | 26 | Lactation cessation | No response in 14 days, + resolution in | OB |
| | | | 30 days | |
| #18 | 28 | Lactation cessation | No response in 14 days, + resolution in | OB |
| | | | 30 days | |

Strength About 15%

| | | | | |
|---|---|---|---|---|
| #19 | 24 | Lactation cessation | + response in 14 days, continued for 3 | OB |
| | | | more days until cream gone | |
| #20 | 25 | Lactation cessation | + response in 16 days | OB |
| #21 | 24 | Lactation cessation | + response in 12 days | OB |
| #22 | 27 | Lactation cessation | + response in 2 weeks | OB |
| #23 | 32 | Lactation cessation | No response in 7 days, switched to | OB |
| | | | 30%, + response in 2 days | |

One embodiment of the invention is a composition for the enhanced delivery of a cabbage composition for the treatment of engorgement through a body surface, comprising a formulation of: (a) a therapeutically effective amount of the cabbage extract; (b) a pharmaceutically acceptable inorganic or organic base with liposomes in an amount effective to provide a pH within the range of about 8.0-13.0 at the localized region of the body surface during administration of the cabbage composition and to enhance the flux of the cabbage composition through the body surface without causing damage thereto; and (c) a pharmaceutically acceptable carrier suitable for topical or transdermal drug administration, such as a liposome dose. The formulation is typically, but not necessarily, an aqueous formulation. The pH is more preferably about 8.0-11.5, and most preferably about 8.5-10.5.

### Example 2

Inflammation/Pain Formula: A similar formulation of cabbage extract and base cream with liposomes was used to reduced inflammation and pain in patients suffering from the same specific symptoms. About 15 to about 30 ml (about 75%) Cabbage Juice Extract as processed above, was mixed with about 30 gm of base cream with liposomes forming the inflammation/pain formula. This formula was applied to swollen or inflamed area of soreness, (i.e. joints such as the elbow, knee, ankle, hip, or wrists/fingers) in patients about three to four times daily until pain is gone or decreased to a manageable state. Pain was not completely eliminated in patients with arthritis, however, there was a decrease in the inflammation in the area of pain. Although not wanting to be bound by theory, the cabbage extract and base cream having liposomes allows for a decrease in the concomitant dosing frequency of oral non-steroidal anti-inflammatory drugs thereby leading to a potential decrease in the side effects of the oral NSAID drugs. This allows for successful NSAID therapy when in combination with the cabbage extract and base cream having liposomes.

The results of treating patients with the cabbage extract having liposomes and base cream for inflammation and pain are shown in Table 2. As a result of using the cabbage extract with liposomal base cream, inflammation and pain was suppressed whether it was due to drug/disease induced galactorrhea or from pregnancy. Furthermore, pain in and around the swollen areas where applied was decreases because the swelling was decreased. The swelling and/or inflammation usually results in about 7 to about 10 days of application depending on the location. The thinner the epidermal layer the quicker the relief.

TABLE 2
INFLAMMATION RESULTS:
Strength About 20%

| | | | |
|---|---|---|---|
| #1 | 1 | Knee swelling decreased | + response in 7 days Pain |
| | | | free/swelling gone |
| #2 | 2 | Elbow pain | + response in 8 days Pain |
| | | | free/continues to use, possibly |
| | | | arthritic also takes Motrin only |
| | | | once daily now, rather than three/day |

One skilled in the art readily appreciates that this invention is well adapted to carry out the objectives and obtain the ends and advantages mentioned as well as those inherent therein. Thus, it should be evident that a composition of cabbage extract in combination with a liposome cream can be used a method to reduce the symptoms of engorgement and variations of the composition and methods are encompassed by the invention. For example, techniques may change as manufacturing of larger quantities of the composition are needed, such industrial scaling of composition production are understood to be within the spirit of the invention. The materials, methods, procedures and techniques described herein are presently representative of the preferred embodiments and are intended to be exemplary and are not intended as limitations of the scope. It is understood that one of ordinary skill in the art of pharmaceutical sciences would have available many pharmaceutical reference books, such as Remmington's Pharmaceutical Sciences 17^{th} Edition. Alfonso Gennaro editor, Mack Publishing Company Easton, Pa. 18042, that would allow one to modify and change formulations for the compositions and method of this invention. As such, changes therein and other uses will occur to those skilled in the art which are encompassed within the spirit of the invention or defined by the scope of the pending claims.

### REFERENCES CITED

The following references, to the extent that they provide exemplary procedural or other details supplementary to those set forth herein, are specifically incorporated herein by reference.

United States Patent Application Publication US 2004/0258778A1 (Ser. No. 10/814,607), titled “Lactation Cessation and Breast Engorgement compositions and Methods of Use,” with Farmer et al., listed as inventors, was published on Dec. 23, 2004.

### REFERENCES

1. Snowden, H M, Renfrew, M J, Woolridge, M W. Treatments for breast engorgement during lactation. Cochrane Database Syst Rev 2001; CD000046.
2. Roberts, K L, Reiter, M, Schuster, D. Effects of cabbage leaf extract on breast engorgement. J Hum Lact 1998; 14:231.
3. Transfer of drugs and other chemicals into human milk. Pediatrics 2001; 108:776.
4. Berens, P D. Prenatal, intrapartum, and postpartum support of the lactating mother. Pediatr Clin North Am 2001; 48:365.
5. Lawrence, R A, Lawrence R M. Breastfeeding: A Guide for the Medical Professions, 5th ed, Mosby, St Louis 1999.
6. Remmington's Pharmaceutical Sciences 17^{th} Edition. Alfonso Gennaro editor, Mack Publishing Company Easton, Pa. 18042, Entire Book, pages 1-1983.
7. Remmington's Pharmaceutical Sciences 17^{th} Edition. Alfonso Gennaro editor, Mack Publishing Company Easton, Pa. 18042, Chapter 68, pages 1278-1321.
8. Remmington's Pharmaceutical Sciences 17^{th} Edition. Alfonso Gennaro editor, Mack Publishing Company, Easton, Pa. 18042, Chapter 84, pages 1492-1517.

What is claimed:

1) A composition comprising:
(a) an effective amount of extracted cabbage juice; and
(b) a base cream containing liposomes;
wherein, the extracted cabbage juice is a product from the inner leaves of a cabbage head.

2) The composition of claim 1, wherein a ratio of extracted cabbage juice in milliliters (“ml”) to base cream containing liposomes in grams (“gm”) is about 1 ml:30 gm.

3) The composition of claim 2, wherein a ratio of extracted cabbage juice in milliliters (“ml”) to base cream containing liposomes in grams (“gm”) is about 5 ml:30 gm.

4) The composition of claim 2, wherein a ratio of extracted cabbage juice in milliliters (“ml”) to base cream containing liposomes in grams (“gm”) is about 10 ml:30 gm.

5) The composition of claim 2, wherein a ratio of extracted cabbage juice in milliliters (“ml”) to base cream containing liposomes in grams (“gm”) is about 15 ml:30 gm.

6) The composition of claim 2, wherein a ratio of extracted cabbage juice in milliliters (“ml”) to base cream containing liposomes in grams (“gm”) is about 20 ml:30 gm.

7) The composition of claim 2, wherein a ratio of extracted cabbage juice in milliliters (“ml”) to base cream containing liposomes in grams (“gm”) is about 25 ml:30 gm.

8) A method of resolving a symptom of breast engorgement, galactorrhea, and other associated disorders in a patient suffering symptoms of breast engorgement, galactorrhea, and other associated disorders comprising:
(a) applying a composition containing cabbage juice and base cream having liposomes to a defatted portion of each breast of the patient; and
(b) repeating step (a) at least once a day until the symptom of breast engorgement, galactorrhea, and other associated disorders has been resolved.

9) The method of claim 8, further comprising: selecting the patient suffering with a nipple leakage symptom of breast engorgement, galactorrhea, and other associated disorders before step (a).

10) The method of claim 8, further comprising: selecting the patient suffering with a breast pain symptom of breast engorgement, galactorrhea, and other associated disorders before step (a).

11) The method of claim 8, further comprising: selecting the patient suffering with a lactation cessation symptom of breast engorgement, galactorrhea, and other associated disorders before step (a).

12) The method of claim 8, further comprising: selecting the patient suffering with a galactorrhea symptom of breast engorgement, galactorrhea, and other associated disorders before step (a).

13) The method of claim 8, further comprising: repeating step (b) for about 1 to about 21 days.

14) The method of claim 8, further comprising: selecting a composition containing extracted cabbage juice and base cream having liposomes, wherein a ratio of extracted cabbage juice in milliliters (“ml”) to base cream containing liposomes in grams (“gm”) is about 1-25ml extracted cabbage juice to about 30 gm base cream containing liposomes.

15) A method of resolving a symptom inflammation or pain in a joint of a patient suffering symptoms of joint pain comprising:
(a) applying a composition containing cabbage juice and base cream having liposomes to the joint of the patient suffering symptoms of joint pain; and
(b) repeating step (a) at least once a day until the symptom of joint pain has been resolved.

16) The method of claim 15, further comprising: selecting the patient suffering with elbow pain before step (a).

17) The method of claim 15, further comprising: selecting the patient suffering with knee pain before step (a).

18) The method of claim 15, further comprising: selecting the patient suffering with ankle pain before step (a).

19) The method of claim 15, further comprising: selecting the patient suffering with hip pain before step (a).

20) The method of claim 15, further comprising: selecting the patient suffering with wrist pain before step (a).

21) The method of claim 15, further comprising: selecting the patient suffering with finger pain before step (a).

22) The method of claim 15, further comprising: repeating step (b) for about 1 to about 21 days.

23) The method of claim 15, further comprising: selecting a composition containing extracted cabbage juice and base cream having liposomes, wherein a ratio of extracted cabbage juice in milliliters (“ml”) to base cream containing liposomes in grams (“gm”) is about 1-25 ml extracted cabbage juice to about 30 gm base cream containing liposomes.
